(19) United States
(12) Patent Application Publication
(10) Pub. No.: US 2021/0330596 A1
(43) Pub. Date: Oct. 28, 2021

(54) PHARMACEUTICAL COMPOSITION FOR USE IN A COMBINATION WITH CANNABINOIDS TO ENHANCE BENEFITS OF CANNABINOIDS TO SUPPORT HUMAN ENDOCANNABINOID SYSTEM

(71) Applicant: Jorge A. Mendoza Montoya, Tegucigalpa (HN)

(72) Inventor: Jorge A. Mendoza Montoya, Tegucigalpa (HN)

(21) Appl. No.: 17/370,767

(22) Filed: Jul. 8, 2021

Related U.S. Application Data
(60) Provisional application No. 63/064,791, filed on Aug. 12, 2020.

(51) Int. Cl. A61K 9/50; A61K 36/185; A61K 45/06; A23L 33/105

(52) U.S. Cl. CPC A61K 9/5089; A61K 9/5036; A23L 33/105; A61K 45/06; A61K 36/185

(57) ABSTRACT

The embodiments herein provide a pharmaceutical composition for use in a combination with cannabinoids that includes a primary essential oil, and a secondary essential oil to support the human endocannabinoid system. The primary essential oil replenishes a plurality of first micronutrients lost during the processing of cannabis. The primary essential oil contains a first therapeutically effective amount of crude cannabis essential oil. The secondary essential oil replenishes a plurality of second micronutrients lost during the processing of cannabis. The secondary essential oil contains a second therapeutically effective amount of crude cannabis essential oil. The primary essential oil and the secondary essential oil are in a liquid form. The liquid form of the primary essential oil and the secondary essential oil are respectively transformed into a powder form by performing a microencapsulation process.

### CROSS-REFERENCE TO RELATED APPLICATIONS

The embodiments herein claim the priority of the US Provisional Patent Application (PPA) filed on Aug. 12, 2020, with the Ser. No. 63/064,791 with the title, “An essential-oil enriched composition for use in direct or in indirect combination with cannabinoids”, and the contents of which are included in entirety as reference herein.

### BACKGROUND

### Technical Field

The embodiments herein are generally related to the field of a pharmaceutical composition. The embodiments herein are particularly related to a pharmaceutical composition for use in a combination with cannabinoids. The embodiments herein are more particularly related to the use of the pharmaceutical composition to enhance the benefits of cannabinoids to support the human endocannabinoid system.

### Description of the Related Art

Generally, cannabis loses a large percentage of its micronutrients during the cannabis confection process. The cannabis confection process includes various stages such as harvesting, curing, extraction, formulation, and concentration process. Examples of the micronutrients lost include but are not limited to terpenes, amino acids, carbohydrates, etc. Typically, these micronutrients provide synergy to cannabinoids for a fuller experience. Some of these micronutrients work in synergy with cannabis cannabinoids such as THC and CBD to produce fuller effects. Examples of the fuller effects are a) faster onset of the effects; b) the increased magnitude of the effects; c) longer duration of the effects and d) decrease unwanted side effects and e) a combination of one or all of the above. Even though these micronutrients have no psychoactive or medical value in and of themselves, in the absence of these micronutrients, the opposite is correct, the onset of effects take longer, the magnitude of the effects is smaller and the duration of the effects is shorter.

US patent publication 20150057342 filed by KOREN; Zohar; et al. discloses a solid self-emulsifying cannabinoid composition that includes cannabinoid, essential oil, or at least one terpene, two emulsifiers, and adsorbing powder. The cannabinoid(s) are essentially solubilized in the mixture of terpene(s), essential oil(s), and at least two emulsifiers. The resulting mixture is adsorbed onto the adsorbing powder(s) affording a solid self-emulsifying composition.

U.S. Pat. No. 10,406,186 issued to Finley, et al. discloses cannabis oil extracts and compositions that include cannabis oil compositions containing vitamin E, and methods for preparing the extracts and compositions. The invention provides a method for preparing a cannabis oil extract comprising eluting cannabinoids from cannabis plant material with a solvent to produce an eluate, filtering the eluate with a filter to produce a filtrate, evaporating the solvent from the filtrate with a distiller to produce a distillate, and purging the distillate under conditions sufficient to remove residual solvent, thereby preparing the extract. The method further includes mixing a quantity of vitamin E with the extract to produce a cannabis oil composition.

This specification recognizes there is a need for a pharmaceutical composition which can be used as a dietary supplement in a combination with cannabinoids to provide a complete and fuller experience to a cannabis user as measured by a faster onset time, increased magnitude of the effects, longer duration of the effects, decreased unwanted side effects.

The above-mentioned shortcomings, disadvantages, and problems are addressed herein and which will be understood by reading and studying the following specification.

### SUMMARY

The various embodiments herein provide a pharmaceutical composition for use in a combination with cannabinoids. According to an embodiment herein, the use of a pharmaceutical composition to enhance the benefits of cannabinoids to support a human endocannabinoid system is provided.

According to an embodiment herein, the pharmaceutical composition for use in a combination with cannabinoids includes a primary essential oil and a secondary essential oil. The primary essential oil replenishes a plurality of first micronutrients lost during the processing of cannabis. The secondary essential oil replenishes a plurality of second micronutrients lost during the processing of cannabis. The primary essential oil and the secondary essential oil are in a liquid form. The liquid form of the primary essential oil and the secondary essential oil are respectively transformed into a powder form by performing a microencapsulation process.

In an aspect, the microencapsulation process is performed by utilizing a food-grade polysaccharide as an encapsulating material.

In an aspect, the microencapsulation process includes a step of micro-spraying the primary essential oil onto 50% of the food-grade polysaccharide to obtain a mixture. Further, the microencapsulation process includes a step of incorporating the secondary essential oil into the mixture and adding the remainder 50% of the food-grade polysaccharide to obtain a final mixture. Furthermore, the microencapsulation process includes a step of adding a food coloring and a flavoring agent to the final mixture.

In an aspect, the food coloring and the flavoring agent is added to the final mixture to obtain a matrix of the primary essential oil, coated by the food-grade polysaccharide, and surrounded by the secondary essential oil covered by the food-grade polysaccharide.

In an aspect, the first therapeutically effective amount of crude cannabis essential oil is at least 1% of the pharmaceutical composition.

In an aspect, the second therapeutically effective amount of crude cannabis essential oil is less than 20% of the pharmaceutical composition.

In an aspect, the pharmaceutical composition is used as a dietary supplement.

In an aspect, the dietary supplement supports an endocannabinoid system of a user.

According to an embodiment herein, the use of a pharmaceutical composition includes at least one primary essential oil and at least one secondary essential oil. The primary essential oil replenishes a plurality of first micronutrients lost during the processing of cannabis. The primary essential oil containing a first therapeutically effective amount of crude cannabis essential oil. The secondary essential oil replenishes a plurality of second micronutrients lost during the processing of cannabis. The secondary essential oil containing a second therapeutically effective amount of crude cannabis essential oil. The primary essential oil and the secondary essential oil are in a liquid form. The liquid form of the primary essential oil and the secondary essential oil are respectively transformed into a powder form by performing a microencapsulation process to obtain the pharmaceutical composition.

In an aspect, the microencapsulation process is performed by utilizing a food-grade polysaccharide as an encapsulating material.

In an aspect, the microencapsulation process includes a step of micro-spraying the primary essential oil onto 50% of the food-grade polysaccharide to obtain a mixture. Further, the microencapsulation process includes a step of incorporating the secondary essential oil into the mixture and adding the remainder 50% of the food-grade polysaccharide to obtain a final mixture. Furthermore, the microencapsulation process includes a step of adding a food coloring and a flavoring agent to the final mixture.

In an aspect, the food coloring and the flavoring agent is added to the final mixture to obtain a matrix of the primary essential oil, coated by the food-grade polysaccharide, and surrounded by the secondary essential oil covered by the food-grade polysaccharide.

In an aspect, the first therapeutically effective amount of crude cannabis essential oil is at least 1% of the pharmaceutical composition.

In an aspect, the second therapeutically effective amount of crude cannabis essential oil is less than 20% of the pharmaceutical composition.

In an aspect, the pharmaceutical composition is used as a dietary supplement.

In an aspect, the dietary supplement supports an endocannabinoid system of a user.

Accordingly, one advantage of the present inventive subject matter is that the pharmaceutical composition enhances the benefits of cannabinoids to support the human endocannabinoid system.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features, and advantages will occur to those skilled in the art from the following description of the preferred embodiment and the accompanying drawings in which:

FIG. 1 illustrates a perspective view of a pharmaceutical composition in a capsule form, according to one embodiment herein.

FIG. 2 illustrates a perspective view of a stereoscopic magnification of the homogeneous oil-polysaccharide-in-oil-polysaccharide microencapsulated powder, according to one embodiment herein.

FIG. 3 illustrates a flowchart of a microencapsulation process, in accordance with an embodiment.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS HEREIN

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical, and other changes may be made without departing from the scope of the embodiments. The following detailed description is therefore not to be taken in a limiting sense.

The various embodiments herein provide a pharmaceutical composition for use in a combination with cannabinoids for the consumption of a cannabis user as a dietary supplement. The pharmaceutical composition is essential oil enriched which can be used as a dietary supplement. The pharmaceutical composition is designed and manufactured to enhance the experience of recreational, occasional, regular, and therapeutic users of cannabis in its various forms. The pharmaceutical composition provides support to the human endocannabinoid system resulting in users of the invention experiencing fuller effects, increased magnitude and extended duration of the effects, and reduction of secondary adverse symptoms or a combination thereof, of the effects cannabis, has on the human body.

According to one embodiment herein, a pharmaceutical composition for use in a combination with cannabinoids is provided. As used herein, the cannabinoid is a chemical compound (such as cannabinol, THC, or cannabidiol in their various forms, isomers, derivatives or variants) that is found in the plant species Cannabis spp. (marijuana), and metabolites and synthetic analogues which may or may not have psychoactive properties. The description highlights several embodiments of the invention provided mainly as examples to better describe the disclosure without limiting the scope of the invention. These embodiments describe different configurations, arrangements, features, and methods along with various figures to schematically describe them. The embodiments are frequently referred to each other, which by itself do not constitute a relationship between them. Moreover, it is possible to combine the features or parts of one embodiment with that of another embodiment in different ways without deviating from the scope of this disclosure.

Also, certain terminologies and naming conventions have been used throughout this description. As one skilled in the art may appreciate, it is possible to use different names to refer to the same idea or feature, which will not limit the scope of the invention, unless specifically mentioned otherwise. The functionality of the various parts of the embodiments should be assigned importance over the names by which they are referred to, as the names by themselves are meant only for differentiation. Numerical values stated in this disclosure may be approximate or exact unless otherwise mentioned specifically and the various embodiments may deviate from the stated numbers without deviating from the intended scope.

In an embodiment, the present invention provides a pharmaceutical composition for use in a combination with cannabinoids, includes a primary essential oil and a secondary essential oil. The primary essential oil replenishes a plurality of first micronutrients lost during the processing of cannabis. In an embodiment, the first therapeutically effective amount of crude cannabis essential oil is at least 1% of the pharmaceutical composition. The secondary essential oil replenishes a plurality of second micronutrients lost during the processing of cannabis. The primary essential oil and the secondary essential oil are in a liquid form. In an embodiment, the second therapeutically effective amount of crude cannabis essential oil is less than 20% of the pharmaceutical composition.

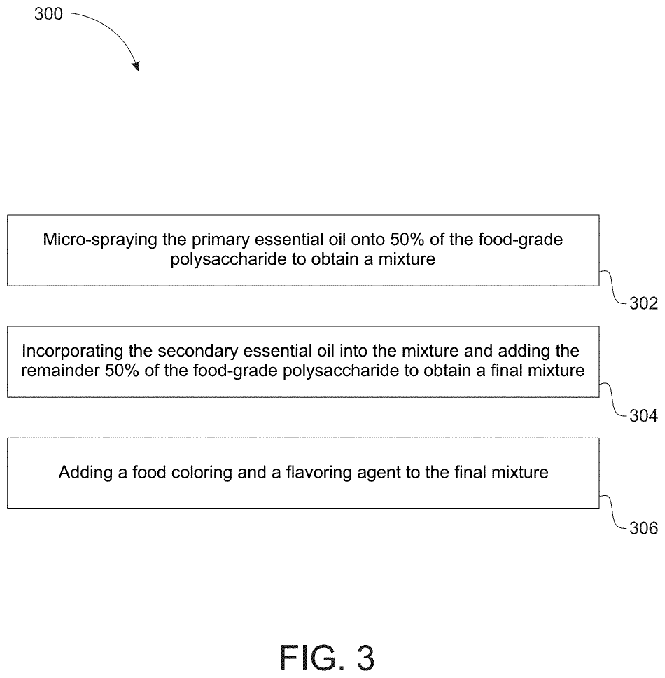

The liquid form of the primary essential oil and the secondary essential oil are respectively transformed into a powder form by performing a microencapsulation process. FIG. 3 illustrates a flowchart 300 of a microencapsulation process, in accordance with an embodiment. In an embodiment, the microencapsulation process is performed by utilizing a food-grade polysaccharide as an encapsulating material. The microencapsulation process includes a step 302 of micro-spraying the primary essential oil onto 50% of the food-grade polysaccharide to obtain a mixture. Further, the microencapsulation process includes a step 304 of incorporating the secondary essential oil into the mixture and adding the remainder 50% of the food-grade polysaccharide to obtain a final mixture. Furthermore, the microencapsulation process includes a step 306 of adding a food coloring and a flavoring agent to the final mixture. In an embodiment, the food coloring and the flavoring agent is added to the final mixture to obtain a matrix of the primary essential oil, coated by the food-grade polysaccharide, and surrounded by the secondary essential oil covered by the food-grade polysaccharide. In an embodiment, the pharmaceutical composition is used as a dietary supplement. In an embodiment, the dietary supplement supports an endocannabinoid system of a user.

In an embodiment, the pharmaceutical composition provides supports to the endocannabinoid system. The human endocannabinoid system is a lipid regulatory system that is present in the brains and bodies of humans. The human endocannabinoid system is involved in physiological and psychological processes, including appetite control, nociception, mood regulation, and the formation and retention of memories. In use, the results of the usage of the present pharmaceutical composition provide a tangible, and measurable variables such as: decreased onset time of the effects (the user feels the effects coming faster) of Tetrahydrocannabinol (THC) and cannabidiol (CBD), increased duration of the effects of THC and CBD; the increased magnitude of the effects of THC and CBD; and decreased side effects of THC and CBD.

According to an embodiment herein, the pharmaceutical composition contains at least one essential oil and may or may not contain cannabis to attain its effect. The pharmaceutical compositions described contains a primary essential oil in a determined quantity and optionally may contain at least one or more essential oils in determined quantities. The embodiments of the pharmaceutical composition may or may not contain cannabis because the pharmaceutical composition is meant to be used in direct, or in indirect combinations with cannabis products.

According to an embodiment herein, the pharmaceutical composition provides at least 20% shorter onset as measured in minutes it takes for the users to experience the effects. Further, the pharmaceutical composition provides an increased magnitude by at least 20% as measured subjectively by each user. Furthermore, the pharmaceutical composition provides a longer duration by at least 20% as reported in minutes by users. Additionally, the pharmaceutical composition provides a decreased side effects by at least 20% as suggested subjectively by each user.

According to an embodiment herein, the present pharmaceutical composition is based on micronutrients selected from essential oils that replicate the micronutrients found in crude cannabis. The pharmaceutical composition provides a cannabis profile in micronutrients from essential oils as close to the original profile as possible.

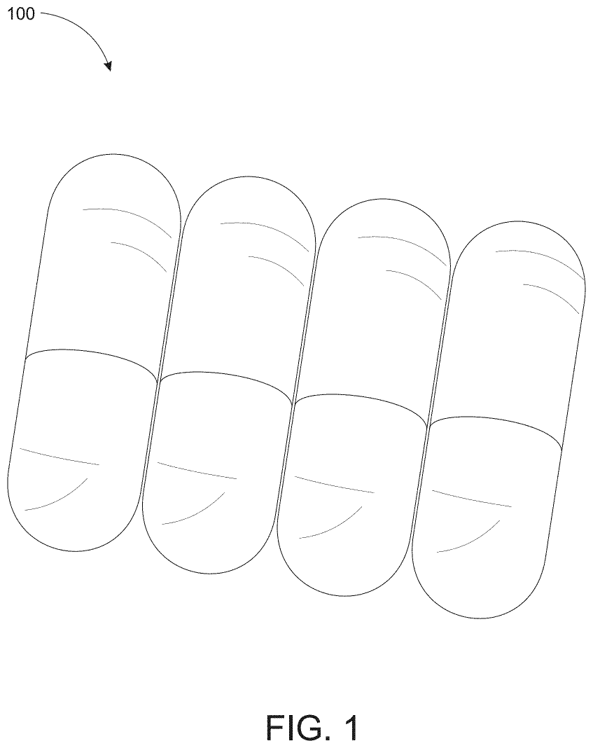

Microencapsulating these micronutrients is key to deliver the correct amount of these micronutrients to the bloodstream at the right time. If the micronutrients are absorbed too soon, they will not be available in the bloodstream when the exocannabinoids are present to deliver the desired fuller effects. Microencapsulation also allows the embodiment to transform essential oils into fine powders. These fine powders are used to procure capsule or tablet formulations. To turn a liquid into a powder, chemists normally evaporate all the water and mill the end resulting paste. But oils and essential oils by definition contain very little to no water. So, microencapsulating the embodiment is a key step to procuring the final pharmaceutical composition viable for use in a dietary supplement such as capsules or tablets shown in FIG. 1. FIG. 1 illustrates a perspective view 100 of a pharmaceutical composition in a capsule form, according to one embodiment herein. The formulation of these natural micronutrients into an embodiment that follows the FDA DHSEA guidelines to procure the dietary supplement for human consumption. The manufacture of the dietary supplements without losing the micronutrients during the production process. The pharmaceutical composition can be packaged in the form of tablets, capsules, gel capsules, patches, suppositories, edibles, beverages, alcoholic beverages, and smoked products such as vape extract. The pharmaceutical composition can also be used concomitantly with cannabis products which means you take the supplement before or after the users take a cannabis product, or may include cannabis products such as cannabis extract itself in the same embodiments mentioned before as part of the formulation.

Typically, a microcapsule is a small sphere of liquid with a uniform wall around it. It is useful to control the release of substances in other media. In pharmaceutics, they are used as control release matrixes to prolong the release of a drug in the bloodstream by protecting the active ingredient with a coating that is slowly dissolved, releasing the active ingredient over time.

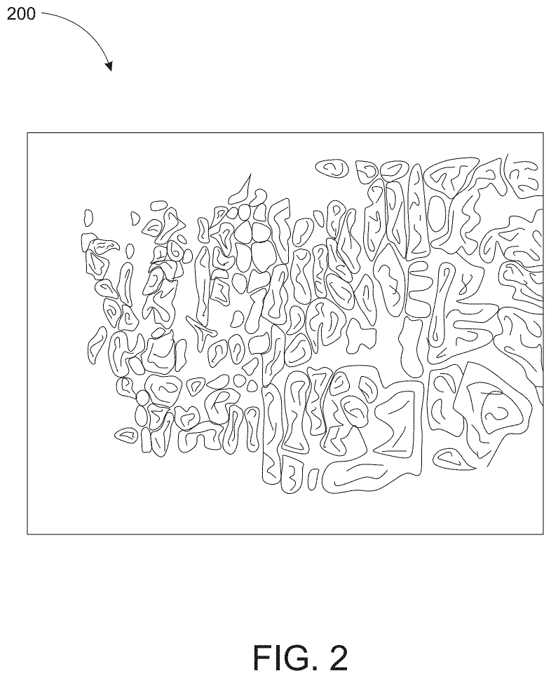

According to an embodiment of the present invention, a micro-droplet is used in a solid matrix technique. A liquid-solid pharmaceutical mixer is capable of spraying droplets of 80-120 μm. In an embodiment, a food grade polysaccharide with 1:10-1:20 absorption capabilities is selected as encapsulating material. The primary essential oil is then micro-sprayed onto 50% the polysaccharide while mixing occurs. Thereafter the secondary essential oil is incorporated into the mix, adding the remainder 50% of the polysaccharide. Finally, food coloring and flavoring agent is added to the mix. The result is a matrix of the primary essential oil, coated by the polysaccharide, surrounded by the secondary essential oil covered by the polysaccharide. Mixing is performed at low-shear (less than 30 rpm) as high-shear will degrade the microcapsules. In operation, heat should be avoided throughout the process as essential oils evaporate even at room temperature. A stereoscopic photograph of the mix is shown in FIG. 2. FIG. 2 illustrates a perspective view 200 of a stereoscopic magnification of the homogeneous oil-polysaccharide-in-oil-polysaccharide microencapsulated powder, according to one embodiment herein. The result is a homogeneous oil-polysaccharide-in-oil-polysaccharide microencapsulated powder.

According to an embodiment herein, the present invention provides the use of a pharmaceutical composition includes at least one primary essential oil and at least one secondary essential oil. The primary essential oil replenishes a plurality of first micronutrients lost during the processing of cannabis. The primary essential oil containing a first therapeutically effective amount of crude cannabis essential oil. In an embodiment, the first therapeutically effective amount of crude cannabis essential oil is at least 1% of the pharmaceutical composition. The secondary essential oil replenishes a plurality of second micronutrients lost during the processing of cannabis. The secondary essential oil containing a second therapeutically effective amount of crude cannabis essential oil. The primary essential oil and the secondary essential oil are in a liquid form. In an embodiment, the second therapeutically effective amount of crude cannabis essential oil is less than 20% of the pharmaceutical composition. The liquid form of the primary essential oil and the secondary essential oil are respectively transformed into a powder form by performing a microencapsulation process to obtain the pharmaceutical composition.

In an embodiment, said non-cannabinoid essential oils may be of the following; Citronella essential oil, Eucalyptus essential oil, Clove oil, Thyme essential oil, Lemmongrass essential oil, Peppermint essential oil, Cinnamon essential oil, Lavender essential oil, Tea Tree essential oil, Rosemary essential oil, Sweet Orange essential oil, Chamommile essential oil, Rose essential oil, Hyssop essential oil, Clay Sage essential oil, Ylang Ylang essential oil, Myrrh essential oil, Vetiver essential oil, Frankinsence essential oil, Helychrysum essential oil, Chrysantemum essential oil, Grapefruit essential oil, Cedarwood essential oil, Pine Tree essential oil, Spearmint essential oil, Basil essential oil, Melaleuca essential oil, Arborvitae essential oil, Cassia essential oil, Oregano essential oil, Agar essential oil, Ajwain essential oil, Angelica root essential oil, Anise essential oil, Asafoetida essential oil, Balsam of Peru essential oil, Bay essential oil, Bergamot essential oil, Black Pepper essential oil, Bachu essential oil, Birch essential oil, Camphor essential oil, Calamodin essential oil, Caraway seed essential oil, Calamus essential oil, Coconut essential oil, Coffee essential oil, Coriander oil, Costmary essential oil, Costus root essential oil, Cranberry seed essential oil, Cubeb essential oil, Cumin seed essential oil, black seed essential oil, Cypress essential oil, Cypriol essential oil, Curry leaf essential oil, Davana essential oil, Dill essential oil, Elecampane essential oil, Elemi essential oil, Fennel seed essential oil, fenugreek essential oil, fir essential oil, Galangal essential oil, Galbanum essential oil, Garlic essential oil, Geranium essential oil, Ginger essential oil, Goldenrod essential oil, Grapefruit essential oil, Henna essential oil, Helichrysum essential oil, Hickory nut essential oil, Horseradish essential oil, Idaho-grown Tansy essential oil, Jasmine essential oil, Juniper berry essential oil, Lemmon essential oil, Laurus nobilis essential oil, Lavender essential oil, Ledum essential oil, Lime essential oil, Litsea cubeba essential oil, Mandarin essential oil, Mango essential oils, Marjoram essential oil, Melissa essential oil (Lemon balm essential oil), Moringa essential oil, Mountain Savory essential oil, Mugwort essential oil, Mustard essential oil, Myrtle essential oil, Neem essential oil, Neroli essential oil, Nutmeg essential oil, Orris essential oil, Palo Santo essential oil, Parsley essential oil, Patchouli essential oil, Perilla essential oil, Petitgrain essential oil, Pineapple essential oil, Ravensara essential oil, Red Cedar essential oil, Rosehip essential oil, Rosewood essential oil, Sandalwood essential oil, Sassafras essential oil, Savory essential oil, Schisandra essential oil, Spikenard Spearmint oil, Spruce essential oil, Star anise essential oil, Tangerine essential oil, Tarragon essential oil, distilled from Artemisia dracunculus, Tsuga essential oil, Turmeric essential oil, Valerian essential oil, Warionia essential oil, Western red cedar essential oil, Wintergreen essential oil and/or Yarrow essential oil, as source of primary or secondary essential oils.

In an embodiment, the microencapsulation process is performed by utilizing a food-grade polysaccharide as an encapsulating material. The microencapsulation process includes a step of micro-spraying the primary essential oil onto 50% of the food-grade polysaccharide to obtain a mixture. Further, the microencapsulation process includes a step of incorporating the secondary essential oil into the mixture and adding the remainder 50% of the food-grade polysaccharide to obtain a final mixture. Furthermore, the microencapsulation process includes a step of adding a food coloring and a flavoring agent to the final mixture. In an embodiment, the food coloring and the flavoring agent is added to the final mixture to obtain a matrix of the primary essential oil, coated by the food-grade polysaccharide, and surrounded by the secondary essential oil covered by the food-grade polysaccharide. In an embodiment, the pharmaceutical composition is used as a dietary supplement. In an embodiment, the dietary supplement supports an endocannabinoid system of a user.

In use, when a cannabis user consumes the pharmaceutical composition in the form of a dietary supplement, he/she can have a fuller experience as measured by shorter onset time, increased magnitude of the effects, and longer duration. Typically, cannabis recreational users are especially fond of the magnitude of the effects so using our embodiment in the form of a dietary supplement will provide increased magnitude while using the same amount of cannabis as always. They can take advantage of using less cannabis than they would normally use, to obtain the same experience. Since the duration of the effects is also extended, they can use fewer repeat doses. For example, some CBD patients take CBD twice a day. During the trials and experiment, CBD users reported only having to use CBD once a day because of the longer duration of the effects. Cannabis medical users can also take advantage of said method embodiment because they can use the embodiment and their cannabis medication to get the desired effects faster, so for example if you take cannabis for pain, the pain relief onset will be faster when they take their cannabis medication concomitantly with our invention.

According to an embodiment herein, the pharmaceutical composition needs the user to take a cannabis product along with the invention for it to be of value. The cannabis product may be taken separately from the pharmaceutical composition or may be taken with the pharmaceutical composition, but the present pharmaceutical composition must be taken with a cannabis product to provide its effects of supporting the human endocannabinoid system.

Further, the present pharmaceutical composition restores as closely as possible the exact cannabis micronutrients that have been lost during the cannabis harvesting, drying, curing, and extraction processes. It thus provides a fuller effect to regular cannabis users. These effects can be described as shorter onset time, increased magnitude of the effects, longer duration of the effects and fewer side effects, or a combination of two or more of these effects.

In an embodiment, the pharmaceutical composition of the present invention relates to an essential oil rich composition that supports the endocannabinoid system. Following tables 1-5 describe trials conducted with ten mice (Wistar) utilizing a maze and food to determine how long on average the mice solves the maze at 10 and 120 minutes after starting the experiment. The results show the effects of the embodiment in combination with and without THC, over the mice's endocannabinoid system.

Table 1 is the base when the mice learned the experiment. It took 7 days of training (4 repetitions per day) to get the base time for each mice.

TABLE 1

| | BASELINE 1 | |
|---|---|---|
| Day 0 | (time in minutes) | |
| Mouse # | Time 0 | Time 1 |
| 1 | 5 ± 1 | 5 ± 1 |
| 2 | 7 ± 1 | 7 ± 1 |
| 3 | 10 ± 1 | 10 ± 1 |
| 4 | 11 ± 1 | 11 ± 1 |
| 5 | 7 ± 1 | 7 ± 1 |
| 6 | 7 ± 1 | 7 ± 1 |
| 7 | 9 ± 1 | 9 ± 1 |
| 8 | 8 ± 1 | 8 ± 1 |
| 9 | 6 ± 1 | 6 ± 1 |
| 10 | 9 ± 1 | 9 ± 1 |
| Average | 8 ± 1 | 8 ± 1 |

Time 0 = 10 minutes after feed
Time 1 = 120 minutes after feed
Dose = 0.0 mgTH/kg (BASELINE)

TABLE 2
each mice were given 0.3 mg of THC
per Kg of body weight. This is the
base-time prior to allowing the mice
to develop tolerance to THC.

| | CONTROL | |
|---|---|---|
| Day 1 | (time in minutes) | |
| Mouse # | Time 0 | Time 1 |
| 1 | 16 ± 3 | 14 ± 3 |
| 2 | 17 ± 3 | 10 ± 3 |
| 3 | 23 ± 3 | 10 ± 3 |
| 4 | 20 ± 3 | 19 ± 3 |
| 5 | 17 ± 3 | 14 ± 3 |
| 6 | 25 ± 3 | 22 ± 3 |
| 7 | 30 ± 3 | 21 ± 3 |
| 8 | 26 ± 3 | 19 ± 3 |
| 9 | 19 ± 3 | 16 ± 3 |
| 10 | 24 ± 3 | 22 ± 3 |
| Average | 22 ± 3 | 17 ± 3 |

Time 0 = 10 minutes after feed
Time 1 = 120 minutes after feed
Dose = 0.3 mgTHC/kg (CONTROL).

Table 3 Shows results after 15 days of daily THC dose of 0.3 mgTHC/kg. FIG. 3 also shows mice starting to solve the maze from 40% faster for TO and up to 60% faster for Ti on average than the Control Time (FIG. 2) demonstrating mice had developed tolerance to THC.

| | BASELINE 2 | |
|---|---|---|
| | (acquired tolerance | |
| Day 14 | in minutes) | |
| Mouse # | Time 0 | Time 1 |
| 1 | 14 ± 3 | 10 ± 3 |
| 2 | 18 ± 3 | 10 ± 3 |
| 3 | 13 ± 3 | 10 ± 3 |
| 4 | 12 ± 3 | 11 ± 3 |
| 5 | 11 ± 3 | 11 ± 3 |
| 6 | 16 ± 3 | 14 ± 3 |
| 7 | 19 ± 3 | 15 ± 3 |
| 8 | 19 ± 3 | 12 ± 3 |
| 9 | 16 ± 3 | 10 ± 3 |
| 10 | 18 ± 3 | 15 ± 3 |
| Average | 14 ± 3 | 12 ± 3 |

Time 0 = 10 minutes after feed
Time 1 = 120 minutes after feed
Dose = 0.3 mgTHC/kg/day (BASELINE2)

Table 4 shows the Trial Results where the 0.6 mg/kg embodiment and the THC 0.3 mg/kg were given to the mice. FIG. 4 also shows mice starting to solve the maze slower than the Control Time (FIG. 3) demonstrating mice had decreased tolerance to cannabis. The Average Time results were statistically slower to the Control (Table 3) results with a standard deviation of less than 3%. The slowness with which the mice solved the maze leads us to believe either the embodiment has some entourage effect (as described by Ben-Shabat et. Al. in the European Journal of Pharmacology, 1998) or has supported the endocannabinoid system in mice because the mice took at least as long to solve the maze as before they developed tolerance to THC. The Entourage Effect was also described by Russo et. al. in his paper “Taming THC: potential cannabis synergy and phytocannabinoid-terpenoid entourage effects”, published in the British Journal of Pharmacology, 2011 August; 163(7): 1344-1364.

| | TRIAL (time | |
|---|---|---|
| Day 15 | in minutes) | |
| Mouse # | Time 0 | Time 1 |
| 1 | 17 ± 3 | 15 ± 3 |
| 2 | 16 ± 3 | 12 ± 3 |
| 3 | 22 ± 3 | 9 ± 3 |
| 4 | 21 ± 3 | 19 ± 3 |
| 5 | 18 ± 3 | 17 ± 3 |
| 6 | 29 ± 3 | 20 ± 3 |
| 7 | 27 ± 3 | 22 ± 3 |
| 8 | 27 ± 3 | 19 ± 3 |
| 9 | 20 ± 3 | 16 ± 3 |
| 10 | 22 ± 3 | 23 ± 3 |
| Average | 22 ± 3 | 17 ± 3 |

Time 0 = 10 minutes after feed
Time 1 = 120 minutes after feed
Dose = 0.3 mgTHC + 0.6 mg embodiment/kg (TRIAL).

Table 5 shows the results of the last experiment; performed to determine whether the embodiment has entourage effect or if it supports the endocannabinoid system, or both. In this Table we see the results of THC 24 hours after the embodiment was administered. Mice on average take 1-5 hours to digest food. It is estimated that mice will have completely metabolized the embodiment after 24 hours. After THC was administered but now without the embodiment, mice are experiencing the same heightened effects of THC as in FIG. 4, even after the embodiment was not present in their body after a 24 hour period. Since mice were solving the maze on average as slow as Table 4, but without the embodiment, we can conclude the embodiment helped mice return to their baseline with regards to their tolerance to cannabis even after 24-hours time lapse, when it is safe to conclude the embodiment is not present in the mice's metabolism; therefore concluding that the embodiment provides endocannabinoid support and not entourage effect, or both, but not entourage effect alone, because if the latter were the case, mice would return to their normal tolerance levels after the embodiment was fully digested.

| | TRIAL (time | |
|---|---|---|
| Day 15 | in minutes) | |
| Mouse # | Time 0 | Time 1 |
| 1 | 17 ± 3 | 15 ± 3 |
| 2 | 16 ± 3 | 12 ± 3 |
| 3 | 22 ± 3 | 9 ± 3 |
| 4 | 21 ± 3 | 19 ± 3 |
| 5 | 18 ± 3 | 17 ± 3 |
| 6 | 29 ± 3 | 20 ± 3 |
| 7 | 27 ± 3 | 22 ± 3 |
| 8 | 27 ± 3 | 19 ± 3 |
| 9 | 20 ± 3 | 16 ± 3 |
| 10 | 22 ± 3 | 23 ± 3 |
| Average | 22 ± 3 | 17 ± 3 |

Time 0 = 10 minutes after feed
Time 1 = 120 minutes after feed
Dose = 0.3 mgTHC + 0.0 mg embodiment/kg (TRIAL 2).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

The embodiments herein provide a pharmaceutical composition for use in a combination with cannabinoids. The pharmaceutical composition is used as a dietary supplement provides a fuller experience to cannabis users as measured by a) faster onset time; b) the increased magnitude of the effects; c) longer duration of the effects and d) decrease unwanted side effects and e) a combination of one or all of the above.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A use of a pharmaceutical composition comprising:
at least one primary essential oil to replenish a plurality of first micronutrients lost during processing of cannabis; and at least one secondary essential oil to replenish a plurality of second micronutrients lost during processing of cannabis, wherein the primary essential oil and the secondary essential oils are in a liquid form, wherein the liquid form of the primary essential oil and the secondary essential oil are respectively transformed into a powder form by performing a microencapsulation process to obtain the pharmaceutical composition.

2. The use of a pharmaceutical composition as claimed in claim 1, to support the human endocannabinoid system when a user consumes the pharmaceutical composition in the form of a dietary supplement, wherein the user has a fuller experience as measured by a shorter onset time, increased magnitude of the effects, and longer duration or a combination of one or all of the above.

3. The use of a pharmaceutical composition as claimed in claim 1, wherein the microencapsulation process is performed by utilizing a food-grade polysaccharide as an encapsulating material.

4. The use of a pharmaceutical composition as claimed in claim 3, wherein the microencapsulation process comprising steps of:
micro-spraying the primary essential oil onto 50% of the food-grade polysaccharide to obtain a mixture;
incorporating the secondary essential oil into the mixture and adding the remainder 50% of the food-grade polysaccharide to obtain a final mixture; and
adding a food coloring and a flavoring agent to the final mixture.

5. The use of a pharmaceutical composition as claimed in claim 4, wherein the food coloring and the flavoring agent is added to the final mixture to obtain a matrix of the primary essential oil, coated by the food-grade polysaccharide, and surrounded by the secondary essential oil covered by the food-grade polysaccharide.

6. The use of a pharmaceutical composition as claimed in claim 1, wherein the first therapeutically effective amount of crude cannabis essential oil is at least 1% of the pharmaceutical composition.

7. The use of a pharmaceutical composition as claimed in claim 1, wherein the second therapeutically effective amount of crude cannabis essential oil is less than 20% of the pharmaceutical composition.

8. The use of a pharmaceutical composition as claimed in claim 1, wherein the pharmaceutical composition obtained from the microencapsulation process is used as a dietary supplement.

9. The use of a pharmaceutical composition as claimed in claim 1, wherein the dietary supplement supports the endocannabinoid system of a user.

10. The use of a pharmaceutical composition as claimed in claim 1, further comprises a plurality of non-cannabinoid essential oils including but not limited to the following: Citronella essential oil, Eucalyptus essential oil, Clove oil, Thyme essential oil, Lemongrass essential oil, Peppermint essential oil, Cinnamon essential oil, Lavender essential oil, Tea Tree essential oil, Rosemary essential oil, Sweet Orange essential oil, Chamomile essential oil, Rose essential oil, Hyssop essential oil, Clay Sage essential oil, Ylang Ylang essential oil, Myrrh essential oil, Vetiver essential oil, Frankincense essential oil, Helychrysum essential oil, Chrysanthemum essential oil, Grapefruit essential oil, Cedarwood essential oil, Pine Tree essential oil, Spearmint essential oil, Basil essential oil, Melaleuca essential oil, Arborvitae essential oil, Cassia essential oil, Oregano essential oil, Agar essential oil, Ajwain essential oil, Angelica root essential oil, Anise essential oil, Asafetida essential oil, Balsam of Peru essential oil, Bay essential oil, Bergamot essential oil, Black Pepper essential oil, Bachu essential oil, Birch essential oil, Camphor essential oil, Calamodin essential oil, Caraway seed essential oil, Calamus essential oil, Coconut essential oil, Coffee essential oil, Coriander oil, Costmary essential oil, Costus root essential oil, Cranberry seed essential oil, Cubeb essential oil, Cumin seed essential oil, black seed essential oil, Cypress essential oil, Cypriol essential oil, Curry leaf essential oil, Davana essential oil, Dill essential oil, Elecampane essential oil, Elemi essential oil, Fennel seed essential oil, fenugreek essential oil, fir essential oil, Galangal essential oil, Galbanum essential oil, Garlic essential oil, Geranium essential oil, Ginger essential oil, Goldenrod essential oil, Grapefruit essential oil, Henna essential oil, Helichrysum essential oil, Hickory nut essential oil, Horseradish essential oil, Idaho-grown Tansy essential oil, Jasmine essential oil, Juniper berry essential oil, Lemmon essential oil, Laurus nobilis essential oil, Lavender essential oil, Ledum essential oil, Lime essential oil, Litsea cubeba essential oil, Mandarin essential oil, Mango essential oils, Marjoram essential oil, Melissa essential oil (Lemon balm essential oil), Moringa essential oil, Mountain Savory essential oil, Mugwort essential oil, Mustard essential oil, Myrtle essential oil, Neem essential oil, Neroli essential oil, Nutmeg essential oil, Orris essential oil, Palo Santo essential oil, Parsley essential oil, Patchouli essential oil, Perilla essential oil, Petitgrain essential oil, Pineapple essential oil, Ravensara essential oil, Red Cedar essential oil, Rosehip essential oil, Rosewood essential oil, Sandalwood essential oil, Sassafras essential oil, Savory essential oil, Schisandra essential oil, Spikenard Spearmint oil, Spruce essential oil, Star anise essential oil, Tangerine essential oil, Tarragon essential oil, distilled from Artemisia dracunculus, Tsuga essential oil, Turmeric essential oil, Valerian essential oil, Warionia essential oil, Western red cedar essential oil, Wintergreen essential oil and/or Yarrow essential oil, as a source of the primary essential oil or the secondary essential oil.

11. A pharmaceutical composition for use in a combination with cannabinoids, comprising:
at least one primary essential oil to replenish a plurality of first micronutrients lost during processing of cannabis, wherein the primary essential oil containing a first therapeutically effective amount of crude cannabis essential oil; and
at least one secondary essential oil to replenish a plurality of second micronutrients lost during processing of cannabis, wherein the secondary essential oil containing a second therapeutically effective amount of crude cannabis essential oil, wherein the primary essential oil and the secondary essential oil are in a liquid form, wherein the liquid form of the primary essential oil and the secondary essential oil are respectively transformed into a powder form by performing a microencapsulation process.

12. The pharmaceutical composition as claimed in claim 11 supports the endocannabinoid system when a user consumes the pharmaceutical composition in the form of a dietary supplement, wherein the user have a fuller experience as measured by a shorter onset time, increased magnitude of the effects, and longer duration or a combination of one or more of the aforementioned effects.

13. The pharmaceutical composition as claimed in claim 11, wherein the microencapsulation process is performed by utilizing a food-grade polysaccharide as an encapsulating material.

14. The pharmaceutical composition as claimed in claim 13, wherein the microencapsulation process comprising steps of:
micro-spraying the primary essential oil onto 50% of the food-grade polysaccharide to obtain a mixture;
incorporating the secondary essential oil into the mixture and adding the remainder 50% of the food-grade polysaccharide to obtain a final mixture; and
adding a food coloring and a flavoring agent to the final mixture.

15. The pharmaceutical composition as claimed in claim 14, wherein the food coloring and the flavoring agent is added to the final mixture to obtain a matrix of the primary essential oil, coated by the food-grade polysaccharide, and surrounded by the secondary essential oil covered by the food-grade polysaccharide.

16. The pharmaceutical composition as claimed in claim 11, wherein the first therapeutically effective amount of crude cannabis essential oil is at least 1% of the pharmaceutical composition.

17. The pharmaceutical composition as claimed in claim 11, wherein the second therapeutically effective amount of crude cannabis essential oil is less than 20% of the pharmaceutical composition.

18. The pharmaceutical composition as claimed in claim 11 is used as a dietary supplement.

19. The pharmaceutical composition as claimed in claim 11, wherein the dietary supplement supports the endocannabinoid system of a user.

20. The use of a pharmaceutical composition as claimed in claim 1, further comprises a plurality of non-cannabinoid essential oils including but not limited to the following: Citronella essential oil, Eucalyptus essential oil, Clove oil, Thyme essential oil, Lemongrass essential oil, Peppermint essential oil, Cinnamon essential oil, Lavender essential oil, Tea Tree essential oil, Rosemary essential oil, Sweet Orange essential oil, Chamomile essential oil, Rose essential oil, Hyssop essential oil, Clay Sage essential oil, Ylang Ylang essential oil, Myrrh essential oil, Vetiver essential oil, Frankincense essential oil, Helychrysum essential oil, Chrysanthemum essential oil, Grapefruit essential oil, Cedarwood essential oil, Pine Tree essential oil, Spearmint essential oil, Basil essential oil, Melaleuca essential oil, Arborvitae essential oil, Cassia essential oil, Oregano essential oil, Agar essential oil, Ajwain essential oil, Angelica root essential oil, Anise essential oil, Asafoetida essential oil, Balsam of Peru essential oil, Bay essential oil, Bergamot essential oil, Black Pepper essential oil, Bachu essential oil, Birch essential oil, Camphor essential oil, Calamodin essential oil, Caraway seed essential oil, Calamus essential oil, Coconut essential oil, Coffee essential oil, Coriander oil, Costmary essential oil, Costus root essential oil, Cranberry seed essential oil, Cubeb essential oil, Cumin seed essential oil, black seed essential oil, Cypress essential oil, Cypriol essential oil, Curry leaf essential oil, Davana essential oil, Dill essential oil, Elecampane essential oil, Elemi essential oil, Fennel seed essential oil, fenugreek essential oil, fir essential oil, Galangal essential oil, Galbanum essential oil, Garlic essential oil, Geranium essential oil, Ginger essential oil, Goldenrod essential oil, Grapefruit essential oil, Henna essential oil, Helichrysum essential oil, Hickory nut essential oil, Horseradish essential oil, Idaho-grown Tansy essential oil, Jasmine essential oil, Juniper berry essential oil, Lemmon essential oil, Laurus nobilis essential oil, Lavender essential oil, Ledum essential oil, Lime essential oil, Litsea cubeba essential oil, Mandarin essential oil, Mango essential oils, Marjoram essential oil, Melissa essential oil (Lemon balm essential oil), Moringa essential oil, Mountain Savory essential oil, Mugwort essential oil, Mustard essential oil, Myrtle essential oil, Neem essential oil, Neroli essential oil, Nutmeg essential oil, Orris essential oil, Palo Santo essential oil, Parsley essential oil, Patchouli essential oil, Perilla essential oil, Petitgrain essential oil, Pineapple essential oil, Ravensara essential oil, Red Cedar essential oil, Rosehip essential oil, Rosewood essential oil, Sandalwood essential oil, Sassafras essential oil, Savory essential oil, Schisandra essential oil, Spikenard Spearmint oil, Spruce essential oil, Star anise essential oil, Tangerine essential oil, Tarragon essential oil, distilled from Artemisia dracunculus, Tsuga essential oil, Turmeric essential oil, Valerian essential oil, Warionia essential oil, Western red cedar essential oil, Wintergreen essential oil and/or Yarrow essential oil, as a source of the primary essential oil or the secondary essential oil.
